(19) United States
(12) Patent Application Publication
(10) Pub. No.: US 2023/0181496 A1
(43) Pub. Date: Jun. 15, 2023

(54) USE OF NOREPINEPHRINE OR BETA-ADRENERGIC RECEPTOR INHIBITOR IN PREPARATION OF MEDICAMENT FOR NERVE INJURY REPAIR IN DIABETES

(71) Applicant: Qingdao Eye Hospital of Shandong First Medical University Shandong Eye Institute, Qingdao Ciity (CN)

(72) Inventors: Qingjun ZHOU, Qingdao City (CN); Ya LI, Qingdao City (CN); Xingyue YUAN, Qingdao City (CN)

(21) Appl. No.: 17/736,257

(22) Filed: May 4, 2022

(30) Foreign Application Priority Data
Dec. 9, 2021 (CN) 202111499055.8

(51) Int. Cl. A61K 31/138; A61K 31/145; A61K 31/132; A61K 31/14; A61K 31/475; A61K 31/135; A61K 31/395; A61P 27/02; A61K 31/165

(52) U.S. Cl. CPC A61K 31/138; A61K 31/145; A61K 31/132; A61K 31/14; A61K 31/475; A61K 31/135; A61K 31/5545; A61P 27/02; A61K 31/165

(57) ABSTRACT

The present disclosure provides use of a norepinephrine or a β-adrenergic receptor inhibitor in the preparation of a medicament for nerve injury repair in diabetes, and belongs to the technical field of nerve repair drugs. By inhibiting norepinephrine secretion or antagonizing β2-adrenergic receptors, the present disclosure promotes the expression of neurotrophic factors in epithelial cells to further regulate nerve repair. Therefore, the present disclosure provides the use of a norepinephrine inhibitor or a β-adrenergic receptor inhibitor in the preparation of a medicament for nerve injury repair in diabetes. The inhibitor provided by the present disclosure features excellent stability and low cost, and has excellent clinical application prospects.

### CROSS REFERENCE TO RELATED APPLICATION

This patent application claims the benefit and priority of Chinese Patent Application No. 202111499055.8, entitled “Use of Norepinephrine or β-Adrenergic Receptor Inhibitor in Preparation of Medicament for Nerve Injury Repair in diabetes” filed on 9 Dec., 2021, the disclosure of which is incorporated by reference herein in its entirety as part of the present application.

### TECHNICAL FIELD

The present disclosure belongs to the technical field of nerve repair drugs, and particularly relates to use of a norepinephrine (NE) or a β-adrenergic receptor inhibitor in the preparation of a medicament for nerve injury repair in diabetes.

### BACKGROUND ART

The nervous system mainly includes central nervous system and peripheral nervous system. The central nervous system mainly includes brain and spinal cords, and some other nerves are known as the peripheral nervous system. Whether peripheral nerve injury can be recovered completely is mainly related to the injury degree. In case of relatively mild peripheral nerve injury, complete recovery can be achieved with timely and effective treatment. In case of severe symptoms of peripheral nerve injury (neurotmesis) or long-term nerve injury, it is difficult to be recovered completely. Therefore, how to promote the regeneration after peripheral nerve injury and restore the function has become a research emphasis increasingly. In the study of the promotion of peripheral nerve regeneration factors, nerve growth factor (NGF) is the nerve cell regulatory factor earliest reported, which plays roles in promoting neuroaxonal regeneration and protecting myelinization and neurons. In addition, gangliosides and basic fibroblast growth factor (bFGF) have also been demonstrated to have diverse effects on promoting nerve growth. However, the efficacy of these NGFs is uncertain, most of which are under investigation and application. Meanwhile, there are still problems about high costs and difficulties in clinical popularization and application.

Cornea is one of the tissues with the densest nerves in the whole body. Corneal nerves include sensory and autonomic nerves. The sensory nerve is a branch originated from the trigeminal ganglion, which radiates into the corneal periphery in a complicated and coordinating manner, penetrates the corneal Bowman's membrane, forms the subbasal plexus, and finally arrives the corneal epithelium. It provides corneal epithelial cells and stromal cells with neurotrophins to maintain corneal transparency and health by mediating tear secretion and corneal reflex. Its nerve terminals carry a plurality of receptors including mechanoreceptors, nociceptors, cold receptors, and chemoreceptors. Corneal nerve damage mainly manifests as hyperesthesia, pain, and hypesthesia, leading to delayed corneal epithelial healing, corneal ulcer, stromal thinning, and tear film change. The cornea is also innervated by the autonomic nerve, which is mainly originated from sympathetic nerve of the superior cervical ganglion and influences cell proliferation and inflammation. There are a plurality of factors causing corneal nerve damage, including corneal surgery, diabetes, infection of pathogenic microorganisms, and a variety of physical, chemical and mechanical factors.

So far, methods of corneal nerve repair mainly include: artificial tears, closure of the lacrimal duct, therapeutic corneal contact lenses, tarsorrhaphy, amniotic membrane transplantation, or corneal transplantation; biological agents such as IGF-1, substance P (SP), neurotrophic factor, semaphorins, vascular endothelial growth factor, and serum and plasma derivatives; and corneal neuralization. There are some limitations in these methods, such as material source, price, surgical trauma, and therapeutic effect. At present, recombinant human nerve growth factor (rhNGF) eye drops, as a special drug for treating moderate and severe neurotrophic keratopathy (NK) approved in the US and Europe, are expected to provide an alternative scheme for surgical intervention. However, this drug also has great limitations in high cost and technical complexity, which restricts wider clinical application. Therefore, seeking for a new, efficient, convenient, and inexpensive treatment method for promoting nerve repair can benefit patients and has a widespread and positive influence and significance in clinical application.

β-Adrenergic receptor antagonists are a group of drugs capable of selectively binding to β-adrenergic receptor to antagonize the activating effect of neurotransmitter and catecholamine on β-receptor. It plays an important role in inhibiting both β1- and β2-receptors simultaneously. β2-Receptors are mainly distributed in effector cell membranes of bronchial smooth muscles, vascular smooth muscles, and corneal epithelium. Agonizing the β2-receptor can dilate the bronchus and vascular smooth muscle, while blocking and antagonizing the β2-receptor can also slow the heart rate and lower blood pressure. However, use of β-adrenergic receptor antagonists in nerve injury repair has not been reported yet.

### SUMMARY

In view of this, an objective of the present disclosure is to provide use of a norepinephrine or a β-adrenergic receptor inhibitor in the preparation of a medicament for nerve repair in diabetes.

The present disclosure provides use of a norepinephrine inhibitor or a β-adrenergic receptor inhibitor in the preparation of a medicament for nerve injury repair in diabetes.

In some embodiments, the nerve includes corneal nerve.

In some embodiments, the medicament is capable of reversing corneal nerve growth inhibition caused by norepinephrine.

In some embodiments, the medicament is capable of reversing downregulated expression of nerve growth factor (NGF) and glial cell line-derived neurotrophic factor (GDNF) in corneal epithelial cells caused by norepinephrine.

In some embodiments, the medicament is capable of promoting diabetic corneal epithelial repair and nerve regeneration.

In some embodiments, the β-adrenergic receptor inhibitor includes a β2-adrenergic receptor inhibitor.

In some embodiments, the β-adrenergic receptor inhibitor is one or more selected from the group consisting of levobunolol, betaxolol, carteolol, sotalol, putrescine dihydrochloride, and ICI 118,551.

In some embodiments, the norepinephrine inhibitor is one or more selected from the group consisting of bretylium, reserpine, 6-hydroxydopamine (6-OHDA, a drug for chemical sympathectomy), and guanethidine.

In some embodiments, the medicament includes topical ophthalmological drugs.

In some embodiments, the norepinephrine inhibitor or the β-adrenergic receptor inhibitor has a concentration of 1-100 μM in the medicament.

The present disclosure provides use of a norepinephrine inhibitor or a β-adrenergic receptor inhibitor in the preparation of a medicament for nerve injury repair in diabetes. Diabetic mice show delayed corneal epithelium and nerve regeneration accompanied by the elevated plasmic and corneal NE contents compared to normal mice. In the present disclosure, β2-adrenergic receptor antagonist (ICI 118,551) or chemical sympathectomy improves corneal nerve growth caused by elevated norepinephrine, which downregulates the expression levels of NGF and GDNF in corneal epithelial cells, and effectively improves diabetic corneal epithelial healing and delayed nerve regeneration. Visibly, the medicament provided by the present disclosure inhibits norepinephrine secretion or antagonizes β2-adrenergic receptors, promotes the expression of neurotrophic factors in epithelial cells, and further regulates nerve repair, exhibiting an excellent repair effect. Meanwhile, the norepinephrine inhibitor or the β2-adrenergic receptor antagonist is a small molecule compound with high purity, excellent stability, and low cost, and has excellent clinical application prospects.

### BRIEF DESCRIPTION OF THE DRAWINGS

FIG. 1 illustrates the epithelial repair in diabetic mice after corneal epithelium scraping in Example 1 of the present disclosure; the left panel illustrates sodium fluorescein staining of diabetic mice after corneal epithelium scraping, and the right panel shows a statistical chart of mouse corneal wound area;

FIG. 2 illustrates the corneal sensitivity at 3, 7, and 10 days after corneal epithelium scraping;

FIG. 3 illustrates the observation of nerve regeneration of controls and diabetic mice under a confocal microscope;

FIG. 4 illustrates the plasmic and corneal norepinephrine content in the control and diabetic mice;

FIG. 5 illustrates the growth status of trigeminal ganglion neurons in different experimental groups, in which the left panel illustrates β III-Tubulin staining of trigeminal ganglion neurons, and the right panel shows a statistical chart of the growth of trigeminal ganglion neurons in different experimental groups;

FIG. 6 illustrates expression levels of neurotrophic factors NGF and GDNF in corneal epithelial cells;

FIG. 7 illustrates the effect of β2-adrenergic receptor antagonist on diabetic corneal epithelial healing, in which the left panel illustrates the sodium fluorescein staining morphology of mice after corneal epithelium scraping, and the right panel shows a statistical chart of mouse corneal wound area;

FIG. 8 illustrates the corneal sensitivity of diabetic mice treated with β2-adrenergic receptor antagonist

FIG. 9 illustrates the promoting effect of β2-adrenergic receptor antagonist on corneal nerve regeneration in diabetic mice;

FIG. 10 illustrates significantly upregulated expression of NGF and GDNF in corneal epithelium by β2-adrenergic receptor antagonist;

FIG. 11 illustrates the effect of 6-OHDA on the corneal epithelium healing and nerve regeneration in diabetic mice, in which the left panel illustrates the sodium fluorescein staining morphologyafter corneal epithelium scraping, and the right panel shows a statistical chart of mouse corneal wound area;

FIG. 12 illustrates the sensitivity of 6-OHDA to diabetic mouse corneal nerve;

FIG. 13 illustrates the promoting effect of 6-OHDA on corneal nerve regeneration in diabetic mice.

### DETAILED DESCRIPTION OF THE EMBODIMENTS

The present disclosure provides use of a norepinephrine inhibitor or a β-adrenergic receptor inhibitor in the preparation of a medicament for nerve injury repair in diabetes.

In some embodiments of the disclosure, the nerve includes corneal nerve. Delayed corneal epithelial healing, slower recovery of corneal sensitivity, and delayed corneal nerve regeneration take place in diabetic mice. Meanwhile, diabetic corneal injury leads to a significant increase in norepinephrine level. In the present disclosure, use of the norepinephrine inhibitor or the β-adrenergic receptor inhibitor reverses the downregulated expression of NGF and GDNF caused by the norepinephrine in corneal epithelial cells, and reversion of corneal nerve growth inhibition caused by norepinephrine promotes corneal epithelium repair and nerve regeneration in diabetes. The norepinephrine inhibitor or the β-adrenergic receptor inhibitor promotes the expression of neurotrophic factors in epithelial cells and further regulates nerve repair.

In some embodiments of the present disclosure, the β-adrenergic receptor inhibitor includes a β2-adrenergic receptor inhibitor. However, because some β-adrenergic receptor inhibitors have inhibitory activity of both β1- and β2-adrenergic receptors, the β-adrenergic receptor inhibitors may exert repair effect. In some embodiments, the β-adrenergic receptor inhibitor is one or more selected from the group consisting of levobunolol, betaxolol, carteolol, sotalol, putrescine dihydrochloride, and ICI 118,551. According to experiments, after mice with corneal injury are treated with β1- and β2-adrenergic receptor inhibitors, respectively, the β2-adrenergic receptor inhibitor rather than β1-adrenergic receptor antagonist can reverse the downregulation of expression levels of NGF and GDNF in corneal cells and the inhibition of nerve growth caused by elevated norepinephrine, and significantly promote diabetic corneal epithelial repair and sensitivity.

In the present disclosure, diabetic corneal nerve injury leads to a significant increase in norepinephrine level, and a high norepinephrine level can inhibit the nerve growth and the expression of neurotrophic factors in corneal epithelial cells. Insome embodiments, the neurotrophic factors are NGF and/or GDNF. Therefore, in the present disclosure, the norepinephrine inhibitor promotesthe diabetic corneal epithelial repair by inhibiting the level of norepinephrine in cells, upregulates the expression levels of NGF and GDNF in the corneal epithelium, and promotes the corneal nerve regeneration and the corneal sensitivity in diabetic mice. In some embodiments, the norepinephrine inhibitor is one or more selected from the group consisting of bretylium, reserpine, 6-OHDA which is a drug for chemical sympathectomy, and guanethidine.

Insom embodiments of the present disclosure, the medicament includes topical ophthalmological drugs or injections. Methods for preparing the topical ophthalmological drugs are not specifically limited in the present disclosure, and methods ofpreparingtopical ophthalmological drugs well known in the art may be used. In some embodiments, the norepinephrine inhibitor or the β-adrenergic receptor inhibitor in the medicament has a concentration range from 1 to 100 μM, in another embodiment 10 to 80 μM, and in a further embodiment from 40 to 60 μM.ICI 118,551 is preferly prepared as topical ophthalmological drugs. The dosage of the ICI 118,551 is as follows: 10 μM, 4 times/day, 5 μL/eye. the 6-OHDA is preferly prepared as injections. The 6-OHDA (100 mg/kg) is dissolved in 0.9% NaCl with 0.02% VC in a ready-to-use manner, intraperitoneally injected for four consecutive days, and a two days interval, and modeled at 7 days.

The use of a norepinephrine or a β-adrenergic receptor inhibitor in the preparation of a medicament for diabetic nerve repair provided by the present disclosure will be described in detail below with reference to examples, but they should not be construed as limiting the protection scope of the present disclosure.

EXAMPLE 1

Epithelial repair and delayed nerve regeneration in diabetic mice after corneal epithlium scraping

Animals were STZ-induced type I diabetic mice (constructedas describedin the prior art: Lingling, Yang, Guohu, Di, Xia, & Qi, et al. (2014). Substance P promotes diabetic corneal epithelial wound healing through molecular mechanisms mediated via the neurokinin-1 receptor. Diabetes, 63(12), 4262-4274.) and their normal controls. Laboratory mice had no corneal defect, neovascularization, or conjunctival injury.

### 1) Regeneration of the Corneal Epithelium

A mouse was given a general anesthesia by the intraperitoneal injection of pentobarbital sodium (50 mg/kg); after local anesthesia with lidocaine eye drops, the epithelium was scraped from a 2 mm region in the middle of the right cornea of the diabetic mouse using a 2 mm trepan and an epithelium scraper, and epithelial defect was observed by sodium fluorescein staining (using Image J software, the area was counted according to the sodium fluorescein staining area).

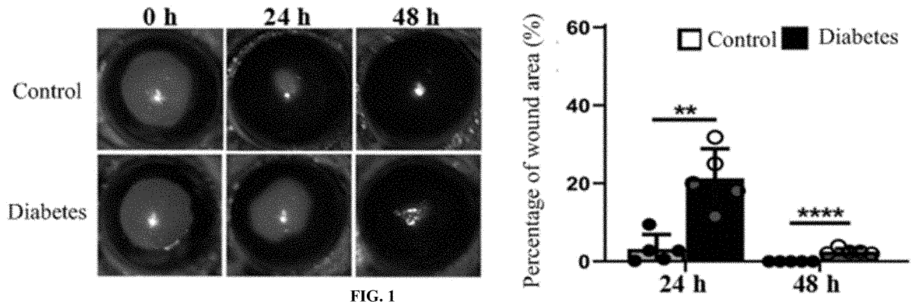

The results are shown in FIG. 1. Results showed that the corneal epithelial regeneration rate was slower in diabetic mice than that in the control. This indicated delayed diabetic epithelial regeneration.

### 2) Recovery Rate of Diabetic Corneal Sensitivity

The corneal sensitivity at 3, 7, and 10 days after corneal epithelial scraping was measured by using a Cochet-Bonnet aesthesiometer, the corneal nerve repair of the diabetic mice was observed, and the corneal sensitivity-time curve was plotted.

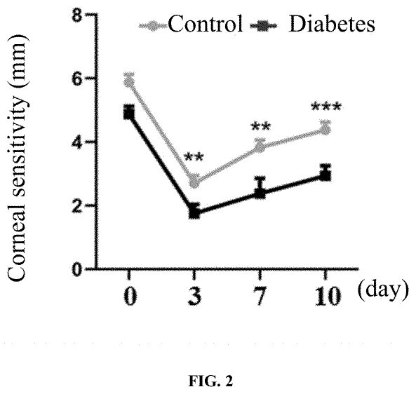

The results are shown in FIG. 2. From FIG. 2, the recovery rate of diabetic corneal sensitivity was significantly slower than that of control mice.

### 3) Corneal Nerve Regeneration

Cornea of diabetic mice and their control mice were subjected to βIII-tubulin staining (as described in the following reference: Wang X, Li W, Zhou Q, et al. MANF Promotes Diabetic Corneal Epithelial Wound Healing and Nerve Regeneration by Attenuating Hyperglycemia-Induced Endoplasmic Reticulum Stress. 2020.), and nerve regeneration was observed under a confocal microscope.

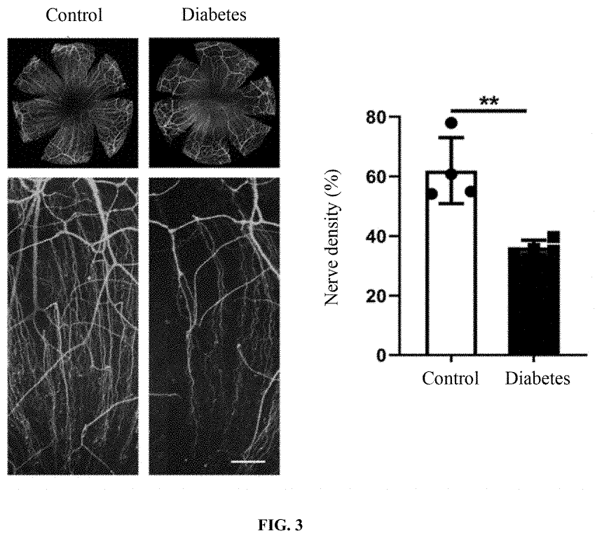

The results are shown in FIG. 3. Delayed corneal nerve regeneration was observed in the diabetic mice.

### EXAMPLE 2

Diabetes-induced elevated levels of norepinephrine in plasma and corneal tissue

The diabetic mice model was established according to the method of Example 1.

After 2 mm corneal epithelial of diabetic and normal mice were scraped, plasma and cornea were collected at 12 and 24 h, respectively, and norepinephrine (NE) levels were detected by using Norepinephrine Enzyme-linked Immunosorbent Assay Kit (Abnova, Cat# ABN-KA3 83 6).

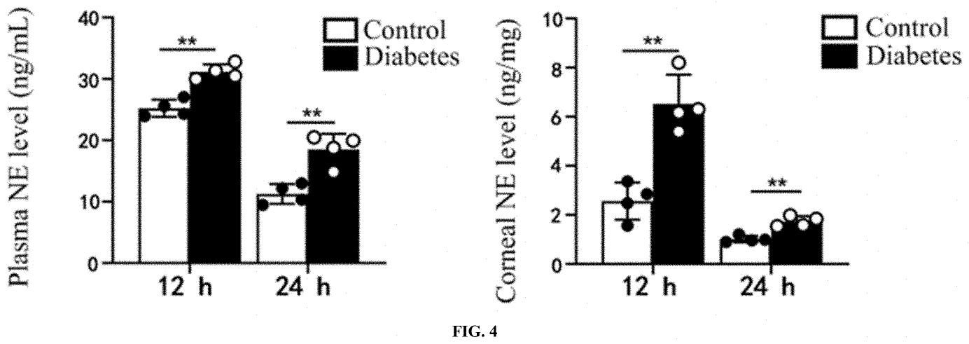

The results are shown in FIG. 4. Corneal injury in the diabetic mice could lead to elevated levels of norepinephrine in plasma and corneal tissue.

### EXAMPLE 3

Improvement of nerve growth inhibition caused by elevated norepinephrine by 132-adrenergic receptor antagonist

TKE2 cells starved for 12 h were treated with 10 μM NE, 10 μM NE+50 μM atenolol (belonging to β1 antagonist), and 10 μM NE+50 μM ICI 118,551 (belonging to β2 antagonist) for 24 h, respectively, while the control group was treated with phosphate-buffered saline (PBS) for 24 h; then, culture supernatants were collected for later use, respectively.

Trigeminal ganglion neurons isolated from normal mice were mixed with a mixture of 40 U/mL papain, 3 mg/mL collagenase, and 4.7 mg/mL dispase II, digested and subjected to gradient centrifugation with percoll (upper layer: 12.5% percoll; lower layer 28% percoll), and isolated to obtain neurons (as describedin the prior art: Malin, S. A., Davis, B. M. & Molliver, D. C. Production of dissociated sensory neuron cultures and considerations for their use in studying neuronal function and plasticity. Nat Protoc 2, 152-160 (2007).). The neurons obtained were cultured on a laminin-coated glass slide overnight. TKE2 culture supernatants under different conditions were added to the neurons and cultured for 24 h; after βIII-Tubulin staining, the length of neuron was analyzed and calculated by Neuron J (after the relative length was analyzed by the software, it was calculated with the control as 1.0).

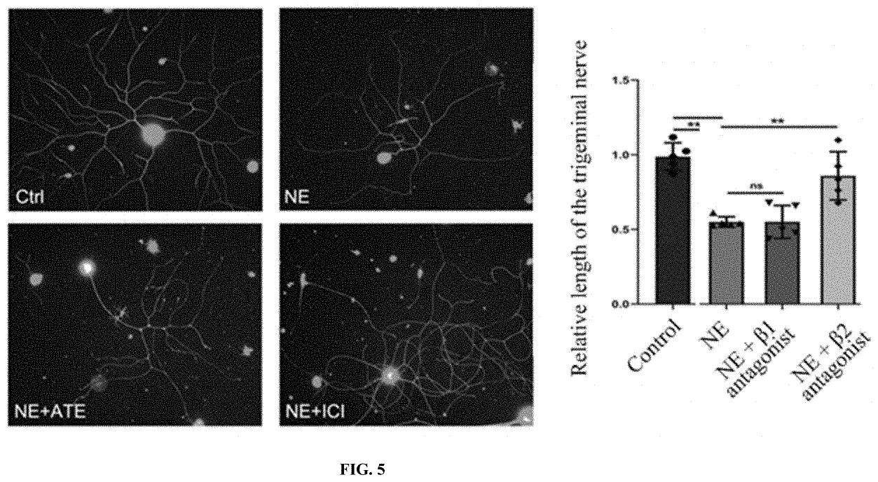

The results are shown in FIG. 5. The β2 antagonist was able to better reverse nerve growth inhibition caused by elevated norepinephrine.

### EXAMPLE 4

TKE2 cells starved for 12 h were treated with 10 μM NE, 10 μM NE+50 μM atenolol, and 10 μM NE+50 μM ICI 118,551 for 24 h, respectively, while the control group was treated with PBS for 24 h; TKE2 cells cultured under different conditions were collected for mRNA extraction, and their culture supernatants were collected; after qPCR and ELISA, expression levels of neurotrophic factors NGF and GDNF were obtained, respectively. Herein, primers for the detection of expression of NGF and GDNF by qPCR were as follows:

| (SEQ ID NO: 1) | |
|---|---|
| NGF: F: | GCCAAGGACGCAGCTTTCTA; |
| (SEQ ID NO: 2) | |
| R: | TTCAGGGACAGAGTCTCCTTCTG; |
| (SEQ ID NO: 3) | |
| GDNF: F: | CAGAGAATTCCAGAGGGAAAGGT; |
| (SEQ ID NO: 4) | |
| R: | CACAGGAACCGCTGCAATATC. |

The reaction system was as follows: 2×SYBR: 5 μL; upstream primer: 0.3 μL; downstream primer: 0.3 μL; RNase-free water: 2.4 μL; cDNA: 2 μL; total: 10 μL;

The reaction program was as follows: PCR initial denaturation: 95° C. for 2 min; two-step program: 40 cycles of 95° C. for 5 s and 60° C. for 10 s.

The ELISA was conducted by ELISA Kit for mouse NGF (USCN Life Science Inc. Wuhan, China) and mouse GDNF(Abcam, ab171178, Shanghai, China).

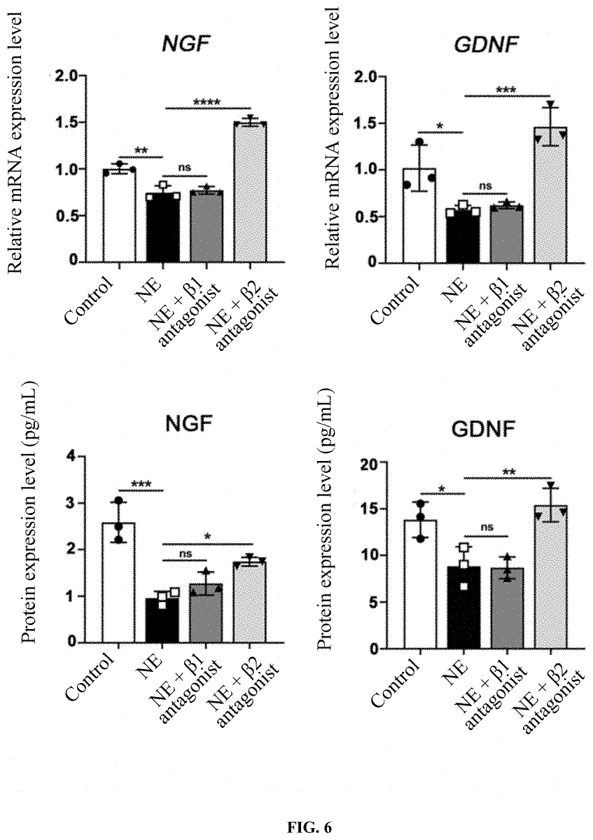

The results are shown in FIG. 6. The β2 receptor antagonist could reverse the downregulated expression of NGF and GDNF caused by NE in TKE2 cells.

### EXAMPLE 5

Improvement of diabetic corneal epithelial healing and delayed nerve regeneration by 132-adrenergic receptor antagonist

The diabetic mice model was established according to the method of Example 1.

After 2 mm corneal epithelium were scraped from diabetic mice, diabetic control mice, and normal control mice, 10-100 μM β2-adrenergic receptor antagonist (5 μL/eye, 4 times/day) was dropped to right eyes of mice for two consecutive days. Saline was dropped to the control mice. Corneal epithelial repair was observed by sodium fluorescein staining at 0, 24, 36, and 48 h after modeling. The corneal sensitivity was measured by using a Cochet-Bonnet aesthesiometer.

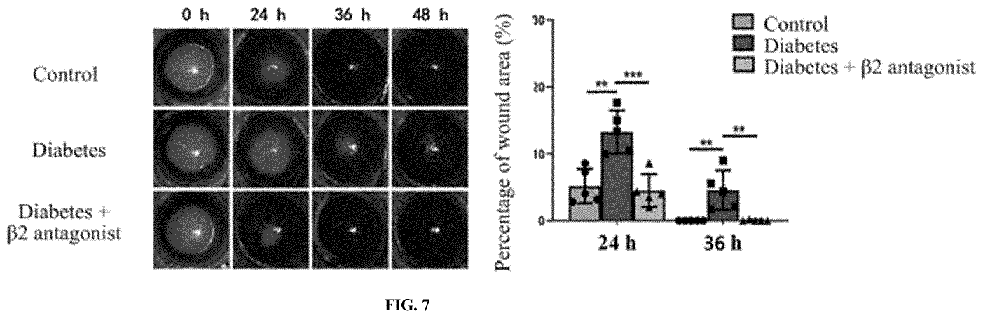

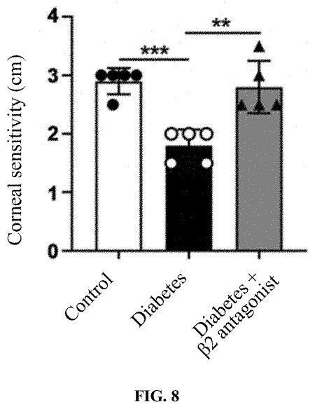

The results are shown in FIGS. 7 and 8. The β2-adrenergic receptor antagonist could significantly promote the diabetic corneal epithelial repair (FIG. 7) and the sensitivity (FIG. 8).

### EXAMPLE 6

The cornea of diabetic mice and their control mice were stained with βIII-tubulin, and nerve regeneration was observed under a confocal microscope.

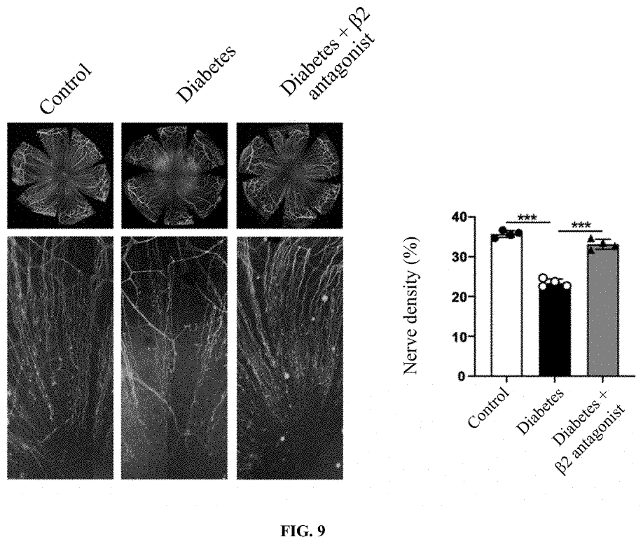

The results are shown in FIG. 9. The β2-adrenergic receptor antagonist could effectively promote corneal nerve regeneration in the diabetic mice.

### EXAMPLE 7

mRNA was extracted from the corneal epithelia of the diabetic mice and the diabetic mice treated with β2-adrenergic receptor antagonist; meanwhile, expression levels of NGF and GDNF in epithelial cells were detected according to the example, and expression levels of NGF and GDNF in epithelia of the diabetic mice and the diabetic mice dropped in eyes with β2-adrenergic receptor antagonist were detected by immunofluorescence staining.

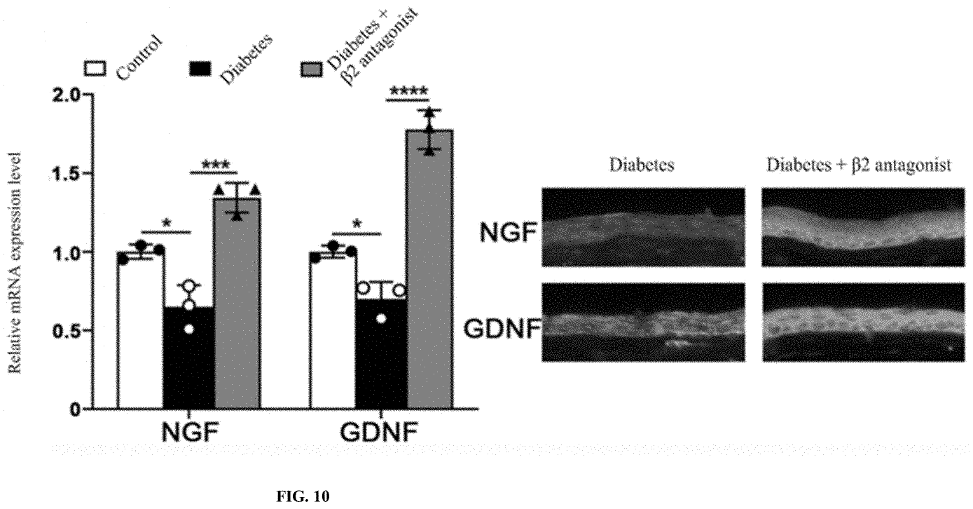

The results are shown in FIG. 10. Both mRNA detection and immunofluorescence staining showed that the β2-adrenergic receptor antagonist significantly upregulated the expression of NGF and GDNF in corneal epithelium. Therefore, the β2-adrenergic receptor antagonist could be used to prepare a medicament for treating delayed healing of diabetic corneal epithelium and nerve regeneration, and the medicament has broad development and application prospects.

### EXAMPLE 8

Improvement of diabetic corneal epithelial healing and nerve regeneration by chemical denervation

1. The diabetic mice model was established according to the method of Example 1.

2. 6-OHDA (100 mg/kg), which is a drug for chemical sympathectomy, was intraperitoneally injected into diabetic mice for four consecutive days in diabetic mice, and saline with 0.02% VC was intraperitoneally injected into control mice. For diabetic mice, diabetic control mice, and normal control mice, 2 mm corneal epithelia were scraped at 7th days, corneal epithelium repair was observed by sodium fluorescein staining at 0, 36, and 48 h after modeling. The corneal sensitivity was measured by using a Cochet-Bonnet aesthesiometer.

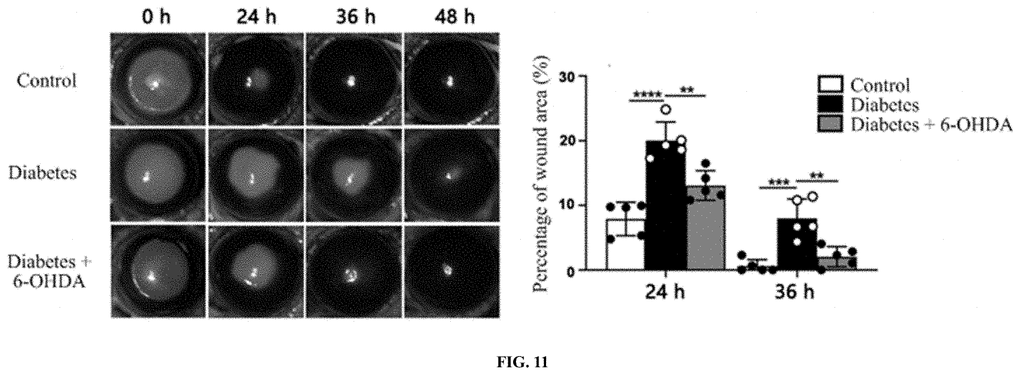

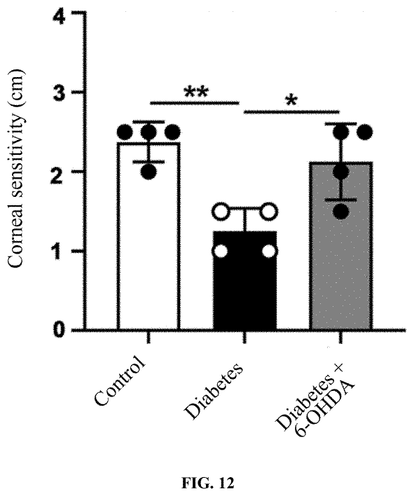

The results are shown in FIGS. 11 and 12. The β2-adrenergic receptor antagonist could significantly promote the diabetic corneal epithelial repair (FIG. 11) and recover the sensitivity (FIG. 12).

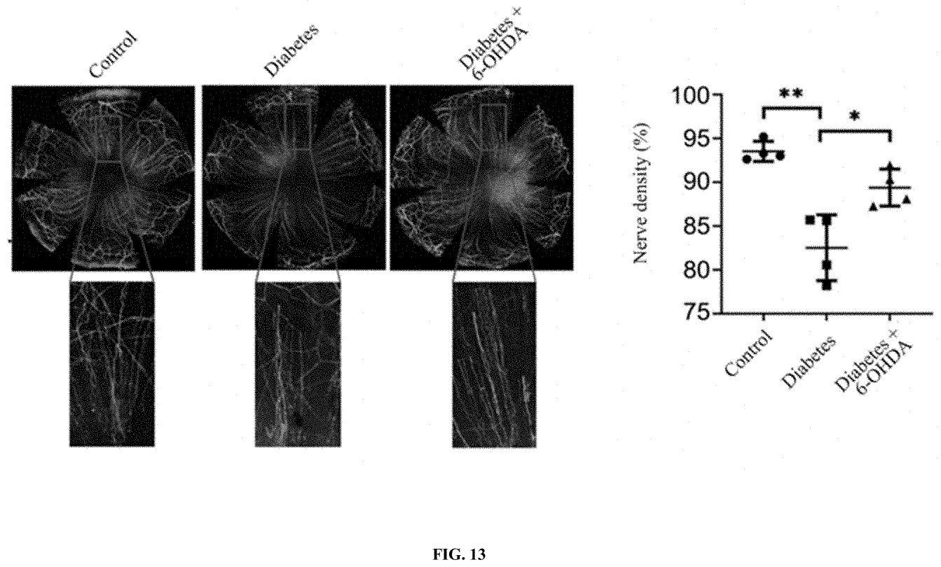

The cornea of diabetic mice and their control mice were stained with βIII-tubulin, and nerve regeneration was observed under a confocal microscope. The results are shown in FIG. 13. The 6-OHDA effectively promoted the corneal nerve regeneration in diabetic mice.

From the results of the foregoing examples, by inhibiting norepinephrine secretion or antagonizing β2-adrenergic receptors, the present disclosure promotes the expression of neurotrophic factors in epithelial cells and further regulates nerve repair. The norepinephrine inhibitor or the β2-adrenergic receptor antagonist is a small molecule compound with high purity, excellent stability, and low cost, and is promising in clinical application.

The above descriptions are merely preferred embodiments of the present disclosure. It should be noted that a person of ordinary skill in the art may further make several improvements and modifications without departing from the principle of the present disclosure, but such improvements and modifications should be deemed as falling within the protection scope of the present disclosure.

1. Use of a norepinephrine inhibitor or a β-adrenergic receptor inhibitor in the preparation of a medicament for nerve injury repair in diabetes.

2. The use according to claim 1, wherein the nerve comprises corneal nerve.

3. The use according to claim 1, wherein the medicament is capable of reversing corneal nerve growth inhibition caused by norepinephrine.

4. The use according to claim 1, wherein the medicament is capable of reversing downregulated expression of nerve growth factor (NGF) and glial cell line-derived neurotrophic factor (GDNF) in corneal epithelial cells caused by norepinephrine.

5. The use according to claim 1, wherein the medicament is capable of promoting diabetic corneal epithelial repair and nerve regeneration.

6. The use according to claim 1, wherein the β-adrenergic receptor inhibitor comprises a β2-adrenergic receptor inhibitor.

7. The use according to claim 1, wherein the β-adrenergic receptor inhibitor is one or more selected from the group consisting of levobunolol, betaxolol, carteolol, sotalol, putrescine dihydrochloride, and ICI 118,551.

8. The use according to claim 1, wherein the norepinephrine inhibitor is one or more selected from the group consisting of bretylium, reserpine, 6-hydroxydopamine (6-OHDA) which is a drug for chemical sympathectomy, and guanethidine.

9. The use according to claim 1, wherein the medicament comprises topical ophthalmological drugs.

10. The use according to claim 9, wherein the norepinephrine inhibitor or the 3-adrenergic receptor inhibitor has a concentration of 1-100 μM in the medicament.

11. The use according to claim 9, wherein the nerve comprises corneal nerve.

12. The use according to claim 9, wherein the medicament is capable of reversing corneal nerve growth inhibition caused by norepinephrine.

13. The use according to claim 9, wherein the medicament is capable of reversing downregulated expression of nerve growth factor (NGF) and glial cell line-derived neurotrophic factor (GDNF) in corneal epithelial cells caused by norepinephrine.

14. The use according to claim 9, wherein the medicament is capable of promoting diabetic corneal epithelial repair and nerve regeneration.

15. The use according to claim 9, wherein the β-adrenergic receptor inhibitor comprises a β2-adrenergic receptor inhibitor.

16. The use according to claim 9, wherein the β-adrenergic receptor inhibitor is one or more selected from the group consisting of levobunolol, betaxolol, carteolol, sotalol, putrescine dihydrochloride, and ICI 118,551.

17. The use according to claim 9, wherein the norepinephrine inhibitor is one or more selected from the group consisting of bretylium, reserpine, 6-hydroxydopamine (6-OHDA) which is a drug for chemical sympathectomy, and guanethidine.


SEQUENCE LISTING (4 sequences)

SEQ ID NO: 1 - DNA, 20 bp, Artificial Sequence
  Forward primer for NGF
gccaaggacgcagctttcta

SEQ ID NO: 2 - DNA, 23 bp, Artificial Sequence
  Reverse primer for NGF
ttcagggacagagtctccttctg

SEQ ID NO: 3 - DNA, 23 bp, Artificial Sequence
  Forward primer for GDNF
cagagaattccagagggaaaggt

SEQ ID NO: 4 - DNA, 21 bp, Artificial Sequence
  Reverse primer for GDNF
cacaggaaccgctgcaatatc